(19) United States
(12) Patent Application Publication
(10) Pub. No.: US 2003/0152643 A1
(43) Pub. Date: Aug. 14, 2003

(54) Combination of an alpha-adrenergic antagonist and a nitrogen oxide donor for treating female sexual dysfunctions

(76) Inventor: Philippe Gorny, Paris (FR)

Correspondence Address: OLIFF & BERRIDGE, PLC, P.O. BOX 19928, ALEXANDRIA, VA, 22320, US

(21) Appl. No.: 09/601,629

(22) PCT Filed: Feb. 12, 1999
(86) PCT No.: PCT/FR99/00318

(30) Foreign Application Priority Data
Feb. 12, 1998 (FR) 98 01690
Sep. 29, 1998 (FR) 98 12172

(51) Int. Cl. A61K 31/542; A61K 31/5377; A61K 31/4745; A61K 31/198; A61K 33/00

(52) U.S. Cl. 424/608; 424/718; 514/283; 514/236.2; 514/225.8; 514/307; 514/565

(57) ABSTRACT

The invention concerns the association of an antagonist of &agr;-adrenergic receptors, for instance yohimbine, and a nitrogen monoxide donor, for instance arginine, with synergistic effect and useful for fighting against female sexual dysfunction.

The invention relates to a medicinal product for preventing or treating certain female sexual dysfunctions.

It is known that, in women, sexual excitation is reflected in particular by a vasodilation of the blood vessels irrigating the genital organs. This vasodilation in particular results in enlargement of the clitoris, as well as vasocongestion of the vaginal walls with exudation of vaginal fluids.

It is also known that, in women, the physiological responses to sexual stimulation can become temporarily impaired, this impairment occasionally being long-lasting, even without any detectable organic cause. The disorders most frequently observed include the absence of sexual desire even after stimulation, difficulty in achieving an orgasm, the low intensity of sexual pleasure and a decrease in or the absence of natural vaginal lubrication, and the consequence of these disorders is often a lack of interest in sexual activity. These disorders in the physiological and/or anatomical response to sexual excitation are referred to in the present application as “female sexual dysfunctions”. According to certain estimates, approximately one woman in three is thought to suffer from such dysfunctions, temporarily or chronically.

It is thus desirable to have available treatments for reducing the seriousness and/or duration of these disorders, or to prevent their occurrence, and to restore the capacity for accomplishment of satisfactory sexual activity, in individuals who present such disorders, or who fear their occurrence.

To this end, the use, mainly in men, of transdermal, transmucous, intranasal or rectal vasodilators (WO 95/05172) or oral vasodilators (WO 96/33705) has been proposed.

Among the vasodilators, a distinction may be made in particular among agents having antagonist effects on the α-adrenergic receptors, which inhibit the adrenergic tonus and thus promote dilation of the arteries, and agents which act as nitrogen monoxide (NO) donors, either directly or in the course of their metabolism. Specifically, it is known that the endothelial cells, which line the inner face of blood vessels, are capable of secreting a substance whose effect is to dilate the arteries, this substance being nitrogen monoxide. It has been established that nitrogen monoxide stimulates the synthesis of cyclic guanosine monophosphate (or cGMP) which is the agent effecting the muscular relaxation of the arteries. It is also known that nitrogen monoxide is the main physiological neurotransmitter used by the non-adrenergic and non-cholinergic peripheral neurons.

Among the α-adrenergic receptor antagonists, also known as “α-blockers”, mention may be made in particular of yohimbine, phentolamine, tolazoline, phenoxybenzamine, chlorpromazine, piperoxan and thymoxamine as well as, where appropriate, the pharmaceutically acceptable salts of these compounds.

Among the products which act on dilation of the arteries by producing nitrogen monoxide, mention may be made, for example, of arginine, sodium nitroprusside, organic nitrates (glyceryl trinitrate and isosorbide mononitrate or dinitrate), organic nitrites (amyl or butyl nitrite), thionitrites such as described in document WO 96/16645 (for example S-nitrosocysteine or S-nitrosoglutathione) and molsidomine as well as, where appropriate, the pharmaceutically acceptable salts of these compounds.

NO donors also exist which additionally have α-blocker activity; these are, in particular, α-blockers substituted with one or more nitro (NO₂) or nitroso (NO) groups, described in document WO 97/27749. The α-blockers which can be substituted with one or more nitro or nitroso groups can be chosen from practically any known α-blocker. These are in particular haloalkylamines such as phenoxybenzamine or dibenzamine; imidazolines such as phentolamine, tolazoline, idazoxan, etc.; quinazolines such as prazosine, terazosine, doxazosine, etc.; indole derivatives such as carvedilol; alcohols such as ifenprodil or labetalol; alkaloids such as ergocornine, ergocristine, ergocryptine, yohimbine, rauwolscine, etc.; or piperidine derivatives such as haloperidol.

It has now been discovered that the combination of two different activities, one being α-adrenergic receptor antagonist activity and the other being nitrogen monoxide donor activity, makes it possible to obtain favorable results in preventing and treating disorders in the physiological and anatomical response to sexual stimulation in women, and thus makes it possible to control said disorders, by virtue of a synergistic effect.

In the present patent application, the expression “nitrogen monoxide donor” means any agent which is capable of directly producing nitrogen monoxide in vivo, any metabolic precursor of such an agent, as well as any agent capable of promoting the endogenous production of nitrogen monoxide.

The invention can be carried out using a medicinal product containing, in combination, an α-adrenergic receptor antagonist and a nitrogen monoxide donor. Needless to say, if an agent having these two types of activity is used, the combination can be reduced to the use of only one agent.

The α-adrenergic receptor antagonists and the nitrogen monoxide donors present in this medicinal product are pharmaceutically acceptable compounds which may chosen in particular from those mentioned above.

The medicinal product of the invention is used so as to administer to the person treated effective doses which may be determined with the aid of suitable tests. It should be pointed out here that many α-blockers and many nitrogen monoxide donors are known, as are their active doses. By comparing the effects of the combination with the effects of each of its active ingredients, it is found that the combination generally makes it possible to reduce the doses of at least one of the active ingredients. Combinations presenting a synergistic effect may thus be selected.

A subject of the invention is also the use, in combination, of an α-adrenergic receptor antagonist and a nitrogen monoxide donor as active ingredients in the preparation of a medicinal product for preventing or treating disorders in the physiological and anatomical response to sexual stimulation in women. This medicinal product is administered to individuals who require it, i.e. to people who have displayed or fear they may have such disorders.

The active ingredients of a medicinal product obtained according to the invention can be presented separately, each in a suitable pharmaceutical form, and combined in the same packaging.

However, to facilitate the simultaneous administration of the active ingredients, it is generally preferred to prepare the medicinal product in a single pharmaceutical form containing the two combined activities (among which is the form of a single active ingredient having these two activities).

The medicinal product of the invention can be administered via the oral, sublingual, nasal, pulmonary, rectal, transmucous or transdermal route.

To this end, it may be provided in any form allowing oral administration (in particular in the form of gel capsules, drinkable solutions or emulsions, powders, gels, granules, lozenges or tablets), nasal administration (for example in the form of solutions to be administered in the form of drops or sprays), pulmonary administration (in particular in the form of solutions in a pressurized bottle for aerosols), rectal administration (suppositories), cutaneous administration (for example ointments or transdermal devices, also known as patches) or transmucous administration such as, for example, sublingual administration (in particular in the form of solutions in a pressurized bottle, or tablets for disintegration in the mouth) or vaginal administration (in particular gynecological creams or pessaries).

These pharmaceutical forms are commonly prepared and can contain suitable conventional excipients and vehicles.

Among the combinations used in accordance with the invention, mention will be made in particular of that of yohimbine with arginine.

Yohimbine is a substance extracted from the bark of the plant Corynanthe yohimbe. It has antagonist properties toward presynaptic alpha-2-adrenergic receptors. In other words, it antagonises the α₂-adrenergic tonus. It has been proposed in men in the treatment of impotence of psychogenic origin and in certain forms of organic impotence, in particular in the case of diabetes. Various side effects such as vertigo, anxiety, nervousness, headaches, insomnia and increase in arterial tension have occasionally been observed, but for relatively high doses of yohimbine; see for example The Medical Letter, French edition, vol. 17, No. 2, 5-6 (ML USA No. 938), 1995.

L-arginine is an endogenous nitrogen monoxide precursor, and its administration is reflected in particular by an effect on the muscular relaxation of the arteries. The administration of L-arginine in men is thought to have a favorable effect on erectile dysfunctions in certain cases; see A. W. Zorgniotti and E. F. Lizza, Int. J. Impotence Res., 6, 33-36 (1994).

Yohimbine and/or arginine can be used in non-salified form, or in salified form.

A medicinal product which can be used according to the invention can be prepared in a pharmaceutical form allowing the administration of a sufficient dose of yohimbine, for example from 2 mg to 10 mg, especially from 2 mg to 8 mg, in particular from 4 mg to 6 mg, in one or two dosage intakes. This dose is calculated as the weight of yohimbine in free base form. The medicinal product is prepared in a pharmaceutical form also allowing the administration of a sufficient dose of arginine which is, for example, a dose of from 1 g to 8 g per day, in particular from 2 g to 6 g, in one or two dosage intakes, the said dose being calculated as the weight of arginine in free base form.

For example, a dose of from 2 mg to 6 mg of yohimbine and from 1 g to 4 g of L-arginine per day can be administered to adults for a treatment needing to last from 2 to 4 weeks. In the case of a spot use, from 4 mg to 10 mg and in particular from 4 mg to 6 mg of yohimbine, and from 2 g to 6 g of L-arginine can be administered, for example, in a single dosage intake, about 1 to 2 hours before envisaged sexual relations.

The examples which follow illustrate the invention.

### EXAMPLES

### Example 1

Gel Capsules

A gel capsule is prepared consisting of a gelatin capsule containing:

| | |
|---|---|
| Arginine: | 0.5 g |
| Yohimbine: | 1 mg |

The yohimbine can be used in free base form or in the form of a salt such as the hydrochloride.

The arginine can be used in free base form or in the form of a pharmaceutically acceptable salt, such as the hydrochloride, glutamate, aspartate or citrate.

In the case of chronic disorders, a systematic daily administration may be envisaged. The duration of the treatment can vary, for example, from 2 to 4 weeks or more.

An episodic use can then be recommended, as and when required.

Similarly, gel capsules are prepared containing either:

| | | |
|---|---|---|
| Yohimbine: | 2 | mg |
| Arginine: | 0.5 | g |
| or: | | |
| Phentolamine mesylate: | 20 | mg |
| Molsidomine: | 2 | mg |

### Example 2

Sachets of Powder for a Drinkable Suspension

Sachets of powder are prepared containing:

| | |
|---|---|
| Yohimbine hydrochloride: | 6 mg |
| Arginine glutamate: | 6 g |
| Flavored excipient: | 3 g |

### Example 3

Tests

Tests were carried out on ten female volunteers, aged from 27 to 60, living in a couple, and suffering from at least one of the following disorders: loss of sexual desire, inability to achieve an orgasm, decrease in the intensity of sexual pleasure, or vaginal dryness. Women whose partners were not themselves suffering from sexual dysfunctions were selected for this test.

The individuals tested were given sachets containing 6 g of arginine glutamate in powder form to be dissolved in water, as well as tablets containing a 2 mg dose of yohimbine, and were requested to simultaneously ingest the contents of one powder sachet along with 3 tablets, 1 to 2 hours before an envisaged sexual activity, and to observe intervals of at least 24 hours between dosage intakes.

The individuals tested evaluated the overall effects observed, after 5 dosage intakes, as regards the following points: sexual desire, achievement of an orgasm, intensity of the sexual pleasure and vaginal lubrication.

Six of the individuals tested found an improvement in at least one of the criteria selected in this study. The other four found no improvement.

In parallel, similar tests were carried out on three women not suffering from any sexual dysfunction. These three women found an increase in the intensity and duration of the orgasms.

1. Use, in combination, of an agent having α-adrenergic receptor antagonist activity and of an agent having nitrogen monoxide donor activity, as active ingredients in the preparation of a medicinal product for preventing or treating disorders in the physiological and anatomical response to sexual stimulation in women.

2. Use according to claim 1, in which said α-adrenergic receptor antagonist is chosen from yohimbine, phentolamine, tolazoline, phenoxybenzamine, chlorpromazine, piperoxan and thymoxamine, and the pharmaceutically acceptable salts thereof.

3. Use according to either of the preceding claims, in which the nitrogen monoxide donor is a compound chosen from arginine, sodium nitroprusside, organic nitrates or nitrites, thionitrites and molsidomine, as well as, where appropriate, the pharmaceutically acceptable salts of these compounds.

4. Use according to any one of the preceding claims, in which the α-adrenergic receptor antagonist is yohimbine.

5. Use according to the preceding claim, in which said medicinal product is prepared in a pharmaceutical form allowing the administration of a dose of from 2 mg to 10 mg, especially from 2 mg to 8 mg and in particular from 4 mg to 6 mg, of yohimbine, in one or two dosage intakes, said dose being calculated as the weight of yohimbine in free base form.

6. Use according to any one of the preceding claims, in which the nitrogen monoxide donor is arginine.

7. Use according to the preceding claim, in which said medicinal product is prepared in a pharmaceutical form allowing the administration of a dose of from 1 g to 8 g and in particular from 2 g to 6 of arginine, in one or two dosage intakes, said dose being calculated as the weight of arginine in free base form.

8. Use according to any one of the preceding claims, in which the α-adrenergic receptor antagonist is yohimbine in free or salified form, and the nitrogen monoxide donor is arginine, in free or salified form.

9. Use according to claim 1, in which said medicinal product contains, as active ingredient, at least one α-blocker, substituted with one or more nitro or nitroso groups, having both α-adrenergic receptor antagonist activity and nitrogen monoxide donor activity.

10. Use according to any one of the preceding claims, in which said medicinal product has at least one of the following characteristics:
it contains said active ingredients separately in the same packaging;
it is in a single pharmaceutical form containing the two active ingredients;
it is in a single pharmaceutical form containing an active ingredient as defined in claim 9;
it is in the form of gel capsules, drinkable solutions or emulsions, granules, gels, powders, lozenges, tablets, ointments, transdermal devices, suppositories, creams or gynecological pessaries, or alternatively in the form of solutions, optionally in pressurized bottles for nasal or pulmonary administration.
